(19) United States
(12) Patent Application Publication
(10) Pub. No.: US 2016/0262740 A1
(43) Pub. Date: Sep. 15, 2016

(54) SURGICAL INSTRUMENT AND METHOD

(71) Applicant: Warsaw Orthopedic, Inc., Warsaw, IN (US)

(72) Inventors: Jason M. May, Cordova, TN (US); William A. Rezach, Atoka, TN (US); Jonathan M. Dewey, North Ryde (AU)

(21) Appl. No.: 14/645,134

(22) Filed: Mar. 11, 2015

(51) Int. Cl. A61B 17/02

(52) U.S. Cl. CPC A61B 17/025; A61B 2017/0256; A61B 17/7074

(57) ABSTRACT

A surgical instrument includes a first member defining a longitudinal axis. A second member is disposed with the first member and is axially translatable relative to the first member. A first body is connected to the first member and is translatable along an arcuate path relative to the first member. The first body is connected to a first implant support. A second body is connected to the second member and is translatable along an arcuate path relative to the second member. The second body is connected to a second implant support. Systems and methods of use are disclosed.

### TECHNICAL FIELD

The present disclosure generally relates to medical devices for the treatment of spinal disorders, and more particularly to a surgical instrument and method for correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. Correction treatments used for positioning and alignment of vertebrae may employ implants, such as, for example, spinal constructs and interbody devices, for stabilization of a treated section of a spine. In some embodiments, the spinal constructs may be manipulated with surgical instruments for compression and distraction of vertebrae. This disclosure describes an improvement over these prior art technologies.

### SUMMARY

In one embodiment, a surgical instrument is provided. The surgical instrument comprises a member defining a longitudinal axis, a first body and a second body. The first body is connected to the member and includes a surface configured to overlap at least a portion of a first end of a first fastener. The first fastener has a second end configured to penetrate tissue, such as, for example, bone. The second body is connected to the member at a different position than the first member and is translatable relative to the first body. The second body includes a surface configured to overlap at least a portion of a first end of a second fastener, such as, for example, a bone screw. The second fastener has a second end configured to penetrate tissue, such as, for example, bone. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a spinal correction system in accordance with the principles of the present disclosure;

FIG. 2 is a perspective view of components shown in FIG. 1;

FIG. 3 is a perspective view of components shown in FIG. 1;

FIG. 4 is a perspective view of components shown in FIG. 1;

FIG. 5 is a perspective view of components of one embodiment of a spinal correction system in accordance with the principles of the present disclosure;

FIG. 6 is a top view of the components shown in FIG. 5;

FIG. 7 is a perspective view of components one embodiment of a spinal correction system in accordance with the principles of the present disclosure; and

FIG. 8 is a perspective view of components of the system shown in FIG. 7 illustrating use.

### DETAILED DESCRIPTION

The exemplary embodiments of the surgical instrument are discussed in terms of medical devices for the treatment of musculoskeletal disorders and more particularly, in terms of a surgical system comprising a surgical instrument and method for correction of a spine disorder. In some embodiments, the system provides a surgical instrument comprising a low profile distractor that can be employed with transforaminal lumbar interbody fusion and/or posterior lumbar interbody fusion surgical procedures.

In one embodiment, the system includes a surgical instrument, such as, for example, a distractor configured for attachment to a first end of a bone fastener, such as, for example, a headless screw shank. In one embodiment, the surgical instrument facilitates visualization while minimizing an implant profile. In one embodiment, the system includes a distractor configured to maintain distraction while a surgeon is performing an interbody implant procedure. In some embodiments, the system includes an adjustable distractor configured to maintain a required distance as the surgeon is distracting a vertebral space with different implant trials. In one embodiment, the system includes a distractor that only requires interaction by the surgeon during initial placement with a surgical site and upon removal from the surgical site.

In one embodiment, the system includes a distractor comprising a rack and pinion distracting mechanism. In one embodiment, the distractor attaches vertically with a screw and pivots medially or laterally for disposal with and capture of the screw with the distractor. This configuration allows the distractor to overlap a portion of the screw, such as, for example, a spherical portion of the screw to prevent accidental disengagement. In one embodiment, the system includes a distractor having a translating end configured to overlap a post of the screw thereby minimizing a length of a rack.

In one embodiment, the system includes a distractor comprising a torsion lock mechanism. In one embodiment, the torsion lock mechanism comprises a caulking gun type of distraction retention.

In one embodiment, the system includes a distractor configured for in situ connection with a screw. In one embodiment, the distractor has a hole for a threaded engagement with an instrument, such as, for example, an inserter that disposes the distractor adjacent a surgical site. In one embodiment, the distractor includes posts for receiving a screw head, such as, for example, a receiver defining an implant cavity, which may be configured for disposal of a spinal rod. In one embodiment, the distractor comprises a member disposed in a rack and pinion engagement upon removal of an inserter. In one embodiment, the distractor is configured for engagement with a first screw and manipulated with the inserter for engagement with a second screw. This configuration distracts vertebrae upon removal of an inserter.

In some embodiments, the inserter resists and/or prevents the ratchet from engaging the rack. For example, only when the inserter is removed, would the distraction mechanism engage.

In one embodiment, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In one embodiment, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context dearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

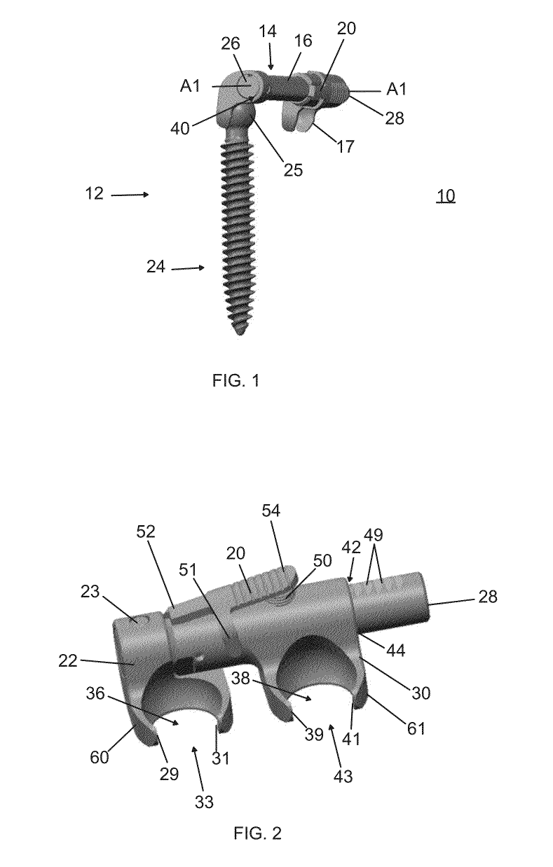

The following discussion includes a description of a surgical system and related methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-4, there are illustrated components of a surgical system, such as, for example, a spinal implant system 10.

The components of system 10 can be fabricated from biologically acceptable materials suitable for medical applications, including metals, synthetic polymers, ceramics, bone material, tissue and/or their composites, depending on the particular application and/or preference of a medical practitioner. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

Spinal implant system 10 is employed, for example, with a fully open surgical procedure, a minimally invasive procedure, including percutaneous techniques, and mini-open surgical techniques to deliver and introduce instrumentation and/or an implant, such as, for example, an interbody implant, at a surgical site within a subject body of a patient, which includes, for example, a spine having vertebrae. In some embodiments, the implant can include spinal constructs, such as, for example, interbody devices, cages, bone fasteners, spinal rods, connectors and/or plates.

System 10 includes a surgical instrument 12 configured for engagement with spinal constructs to correct a spinal disorder, such as, for example, trauma and/or fracture of vertebrae, which may include a sagittal deformity, as described herein. Instrument 12 includes a member, such as, for example, a distraction rack 14. Rack 14 includes a linear shaft 16 defining a longitudinal axis A1. Shaft 16 has an outer surface 18 having a plurality of teeth 49 configured to engage a finger 20, such as, for example a pawl, of a ratchet mechanism, as described herein. In some embodiments, the plurality of teeth 49 are positioned along the entire length of shaft 16. In some embodiments, at least a portion of the length of shaft 16 is free of teeth. In some embodiments, the plurality of teeth 49 span completely around outer surface 18 of shaft 16. In some embodiments, the plurality of teeth 49 span partially around outer surface 18 of shaft 16. In some embodiments teeth 49 are uniform but asymmetrical, with each tooth having a moderate slope on one end and a much steeper slope on the other edges of the tooth. In some embodiments, shaft 16 has a variously configured cross section configuration, such as, for example, cylindrical, oval, oblong, triangular, rectangular, square, polygonal, irregular, uniform, non-uniform, variable and/or tapered.

In some embodiments, rack 14 extends between an end 26 and an end 28 in a substantially linear orientation. A first body member 22 includes a cup-shaped retaining member 60 including an arcuate inner surface 32 defining a cavity 36 configured to mate with a head 25 on a fastener 24, such as, for example, a bone screw. In some embodiments, member 22 is fixed relative to rack 14. In some embodiments, head 25 is spherical or substantially spherical. In some embodiments, head 25 has a variously configured cross section configuration, such as, for example, cylindrical, oval, oblong, polygonal, irregular, uniform, non-uniform, variable and/or tapered. In some embodiments head 25 is spherical and has a maximum diameter greater than that of cavity 36 such that head 25 can pivotally engage surface 32 with a threaded shaft 27 of fastener 24 extending through cavity 36, as shown in FIG. 4, for example. In some embodiments, head 25 includes a plurality of ridges to improve purchase of head 25 with member 22.

Cavity 36 is further defined by opposing end surfaces 29, 31 of member 60. Surfaces 29, 31 define an opening 33 therebetween. Opening 33 has a maximum width or diameter that is less than that of cavity 36 and head 25 has a maximum diameter that is greater than that of cavity 36, as discussed above. This provides a snap-fit configuration in which head 25 can be inserted into cavity 36 in a direction that is perpendicular to axis A1. That is, because head 25 has a maximum diameter that is greater than that of cavity 36, head 25 is prevented from falling through cavity 36 and thus engages surface 32 such that head 25 is rotatable relative to member 60.

In some embodiments, cup-shaped retaining member 60 is lowered down vertically, or top-loaded, onto head 25, snapped together with head 25 and partially rotated underneath head 25 so as to prevent head 25 from sliding out of the retaining member 60. Returning head 25 to the original vertical position allows head 25 to slide out of retaining member 60.

In some embodiments, member 60 comprises a deformable material that allows a width or diameter of opening 33 to increase as head 25 is positioned through opening 33. This allows head 25 to be inserted into cavity 36 in a direction that is perpendicular to axis A1 or substantially perpendicular to axis A1. Once head 25 passes opening 33, shaft 27 extends through cavity 36 in a direction that is perpendicular or substantially perpendicular to axis A1 and an outer surface of head 25 engages surface 32, thus allowing shaft 27 to rotate relative to member 60 in a plurality of planes. Fastener 24 includes a neck 35 positioned between head 25 and shaft 27. Neck 35 has a maximum width or diameter that is less than that of opening 33 to allow neck 35 to be disposed in opening 33, as shown in FIG. 4, for example.

In some embodiments, first body member 22 also includes an inner surface 37 defining a first passageway 40 configured to receive shaft 16 of rack 14. In some embodiments, passageway 40 is configured to completely or incompletely surround shaft 16 such that shaft 16 is axially translatable within passageway 40 along longitudinal axis A1. In some embodiments, shaft 16 is rotatable within passageway 40, but is prevented from translating axially within passageway 40. In some embodiments, the rotational position of member 22 relative to shaft 16 can be provisionally fixed by a pin 23 that extends through member 22 and into shaft 16, as shown in FIG. 2. In some embodiments, member 22 is integrally and/or monolithically formed with shaft 16.

In some embodiments, system 10 includes second body member 30 having a cup-shaped retaining member 61 including an arcuate inner surface 34 defining a cavity 38 configured to mate with a head 46 of a second fastener 48, such as, for example, a bone screw. In some embodiments, head 46 is spherical or substantially spherical. In some embodiments, head 46 has a variously configured cross section configuration, such as, for example, cylindrical, oval, oblong, polygonal, irregular, uniform, non-uniform, variable and/or tapered. In some embodiments, head 46 is spherical and has a maximum diameter greater than that of cavity 38 such that head 46 can pivotally engage surface 34 with a threaded shaft 47 of fastener 48 extending through cavity 38, as shown in FIG. 4, for example.

Cavity 38 is further defined by opposing end surfaces 39, 41 of member 61. Surfaces 39, 41 define an opening 43 therebetween. Opening 43 has a maximum width or diameter that is less than that of cavity 38 and head 46 has a maximum diameter that is greater than that of cavity 38, as discussed above. This provides a snap-fit configuration in which head 46 can be inserted into cavity 38 in a direction that is perpendicular to axis A1.

In some embodiments, retaining member 61 is configured so that when cavity 38 is lowered down vertically onto head 46 and snapped in place, it can be rotated underneath head 46 so as to prevent head 46 from sliding out of cavity 38. Returning head 46 to the original vertical position allows head 46 to slide out of retaining member 61 and disengage.

In some embodiments, member 61 comprises a deformable material that allows a width or diameter of opening 43 to increase as head 46 is positioned through opening 43. This allows head 46 to be positioned in cavity 38 in a direction that is perpendicular or substantially perpendicular to axis A1. In particular, once head 46 passes opening 43, shaft 47 extends through cavity 38 in a direction that is perpendicular or substantially perpendicular to axis A1 and an outer surface of head 46 engages surface 34, thus allowing shaft 47 to rotate relative to member 61 in a plurality of planes. Fastener 48 includes a neck 45 positioned between head 46 and shaft 47. Neck 45 has a maximum width or diameter that is less than that of opening 43 to allow neck 45 to be disposed in opening 43, as shown in FIG. 4, for example.

In some embodiments, second body member 30 also includes an inner surface 44 defining a second passageway 42 configured to slidingly receive shaft 16 of rack 14. In some embodiments, second passageway 42 is configured to completely or incompletely surround shaft 16 such that shaft 16 is axially translatable within passageway 42 along longitudinal axis A1.

In some embodiments, instrument 12 includes a finger 20 having a first end 52 and a second end 54. Finger 20 includes an arm 51 that surrounds at least a portion of member 30, as shown in FIG. 2, for example. In some embodiments, arm 51 is pivotally connected to member 30 such that end 52 can pivot relative to end 54, with arm 51 remaining in engagement with member 30 as ends 52, 54 pivot. In some embodiments, first end 52 is configured to engage teeth 49 so that when teeth 49 move axially in an opposite direction of finger 20, the first end 52 of finger 20 will catch against the steeply slope edge of the first tooth 49 that it encounters, thereby locking finger 20 against the tooth and preventing any further motion. In some embodiments, finger 20 is in contact with a biasing member, such as, for example, a spring 50 that is disposed between finger 20 and shaft 16. The biasing member 50 is positioned so as to bias the first end 52 of finger 20 against teeth 48 and automatically locking finger 20 in place. In some embodiments, instrument 12 is a distractor and locking tooth 49 against finger 20 maintains distraction while a surgeon is performing an interbody implant procedure. In some embodiments, the system 10 includes an adjustable distractor configured to maintain a required distance as the surgeon is distracting a vertebral space with different implant trials.

In assembly, operation and use, a surgical implant system, similar to system 10 described herein, is employed with a surgical procedure for treatment of a spinal disorder affecting a section of a spine of a patient, as discussed herein. For example, system 10 can be used with a surgical procedure for treatment of a condition or injury of an affected section of the spine including vertebrae. In some embodiments, one or all of the components of system 10 can be delivered as a pre-assembled device or can be assembled in situ. System 10 may be completely or partially revised, removed or replaced.

For example, system 10 can be employed with a surgical treatment of an applicable condition or injury of an affected section of a spinal column and adjacent areas within a body, such as, for example, vertebrae (not shown). In some embodiments, system 10 may be employed with one or a plurality of vertebra. To treat a selected section of the vertebrae, a medical practitioner obtains access to a surgical site including the vertebrae in any appropriate manner, such as through incision and retraction of tissues. In some embodiments, system 10 can be used in any existing surgical method or technique including open surgery, mini-open surgery, minimally invasive surgery including percutaneous surgical implantation, whereby the vertebrae are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for delivery of implantable components of system 10 such as, for example, fasteners 24 and 48 and rack 14, as shown in FIG. 4. A preparation instrument (not shown) can be employed to prepare tissue surfaces of vertebrae, as well as for aspiration and irrigation of a surgical region. In one embodiment, the system includes a distractor configured to maintain distraction while a surgeon is performing an interbody implant procedure. In some embodiments, the system includes an adjustable distractor configured to maintain a required distance as the surgeon is distracting a vertebral space with different implant trials. In one embodiment, the system includes a distractor that only requires interaction by the surgeon during initial placement with a surgical site and upon removal from the surgical site.

In operation, to treat a condition of the spine, such as, for example, trauma of the spine, fasteners 24, 48 are inserted into vertebrae such that fasteners 24, 48 are spaced apart from one another. Fasteners 24, 48 are each movable from a first configuration, which may include fasteners 24, 48 being disposed in an initial axial position and in an initial angle relative to one another and a second configuration, which may include fasteners 24, 48 being disposed at a corrected axial position and angle relative to one another.

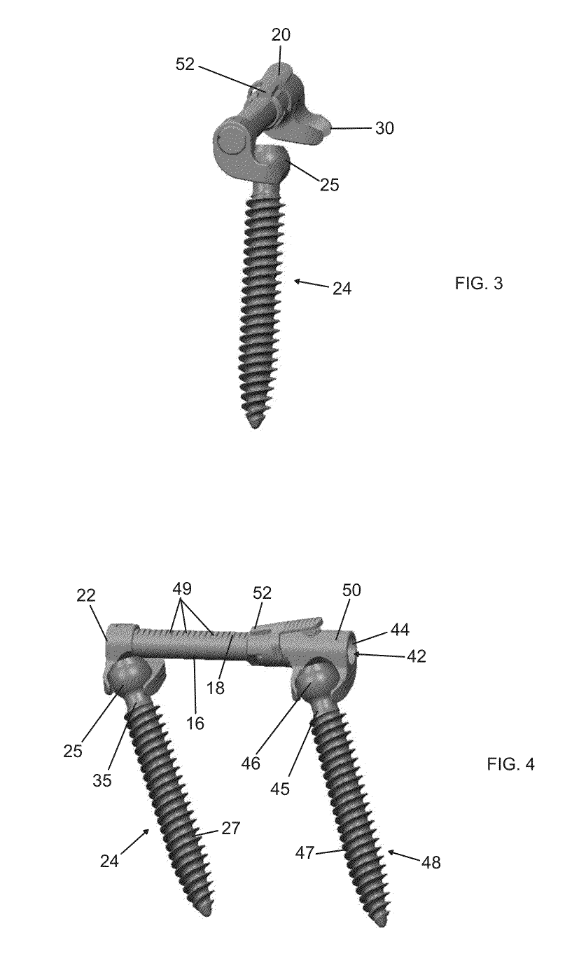

Member 22 is positioned relative to fastener 24 such that cavity 36 extends perpendicular or substantially perpendicular to an axis defined by shaft 27, as shown in FIG. 1. Member 22 is rotated about head 25 such that neck 35 extends through opening 33 and cavity 36 is coaxial or substantially or substantially coaxial with the axis defined by shaft 27, as shown in FIGS. 3 and 4.

A downward force is exerted on end 54 such that end 52 disengages at least one of teeth 49 to allow shaft 16 to slide within passageway 42 along axis A1. Member 30 is moved relative to member 22 along shaft 16 a selected distance from member 22 such that cavity 38 is positioned adjacent head 46. In some embodiments, the selected distance correlates to a desired amount of distraction between vertebrae.

Member 30 is positioned relative to fastener 48 such that cavity 38 extends perpendicular or substantially perpendicular to an axis defined by shaft 47, similar to cavity 36 and shaft 27 in FIG. 1. Member 30 is rotated about head 46 such that neck 45 extends through opening 43 and cavity 38 is coaxial or substantially or substantially coaxial with the axis defined by shaft 47, as shown in FIGS. 3 and 4. Head 46 engages surface 37 to prevent head 46 from falling through cavity 38. The downward force is removed from end 54 such that spring 50 exerts an upward force on end 54, causing end 54 to engage at least one of teeth 49 and fix member 30 relative to member 22 to maintain the selected distance. This configuration allows a medical practitioner to maintain distraction of vertebrae with instrument 12 while he or she performs an interbody procedure. In some embodiments, instrument 12 self-adjusts to maintain the selected distance, and hence distraction of vertebrae.

Surgical instrument 12 may be re-assembled for use in a surgical procedure. In some embodiments, surgical instrument 12 may comprise various instruments including a lock and collet configuration of the present disclosure, with, for example, inserters, extenders, reducers, spreaders, distractors, blades, retractors, clamps, forceps, elevators and drills, which may be alternately sized and dimensioned, and arranged as a kit.

Upon completion of a procedure, surgical instrument 12, surgical instruments and/or tools, assemblies and non-implanted components of system 10 are removed and the incision(s) are closed. One or more of the components of system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of system 10. In some embodiments, system 10 may include one or a plurality of plates, connectors and/or bone fasteners for use with a single vertebral level or a plurality of vertebral levels.

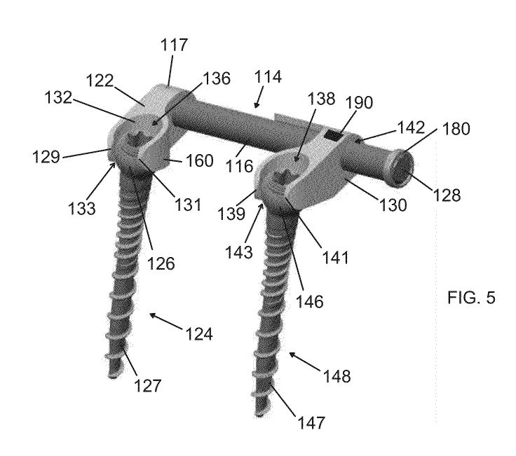

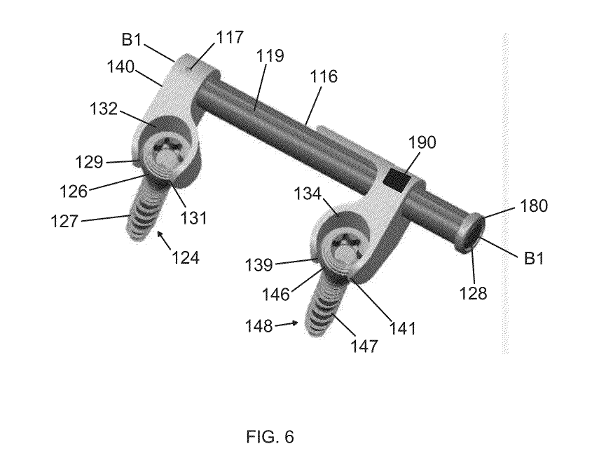

Turning to FIGS. 5 and 6, system 10 includes one or more instruments, such as, for example, instruments 100, similar to instrument 12. Instrument 100 includes a first body member 122, similar to member 22, having a cup-shaped retaining member 160 including an arcuate inner surface 132 defining a cavity 136 configured to mate with a head 126 on a fastener 124, such as, for example, a bone screw similar to fastener 24. In some embodiments, head 126 is spherical or substantially spherical. In some embodiments, head 126 has a variously configured cross section configuration, such as, for example, cylindrical, oval, oblong, polygonal, irregular, uniform, non-uniform, variable and/or tapered. In some embodiments head 126 is spherical and has a maximum diameter greater than that of cavity 136 such that head 126 can pivotally engage surface 132 with a threaded shaft 127 of fastener 124 extending through cavity 136, as shown in FIG. 6, for example. In some embodiments, head 126 includes a plurality of ridges to improve purchase of head 126 with member 122.

Cavity 136 is further defined by opposing end surfaces 129, 131 of member 160. Surfaces 129, 131 define an opening 133 therebetween. Opening 133 has a maximum width or diameter that is less than that of cavity 136 and head 126 has a maximum diameter that is greater than that of cavity 136, as discussed above. This provides a snap-fit configuration in which head 126 can be inserted into cavity 136. In particular, member 160 comprises a deformable material that allows a width or diameter of opening 133 to increase as head 126 is positioned through opening 133. Once head 126 passes opening 133, shaft 127 extends through cavity 136 and an outer surface of head 126 engages surface 132, thus allowing shaft 127 to rotate relative to member 160 in a plurality of planes. In some embodiments, cup-shaped retaining member 160 is lowered down vertically, or top-loaded, onto head 126 and snapped together with head 126 so as to prevent head 126 from sliding out of the retaining member 160.

In some embodiments, first body member 122 also includes an inner surface 137 (not shown), similar to surface 37, defining a first passageway 140 (not shown), similar to passageway 40, configured to receive a shaft 116 of a rack 114 defining a longitudinal axis B1. In some embodiments, passageway 140 is configured to completely or incompletely surround shaft 116 such that shaft 116 is axially translatable within passageway 140. In some embodiments, passageway 140 has various cross sectional configurations, such as, for example, circular, cylindrical, oval, oblong, triangular, square, polygonal, irregular, uniform, non-uniform, offset, staggered, undulating, arcuate, variable and/or tapered. An outer surface 118 of shaft 116 is smooth or even. In some embodiments, surface 118 is free of any gaps or protrusions. In some embodiments, surface 118 defines a groove 119 that extends the entire length of shaft 116. Groove is uniform along the length of shaft 116. In some embodiments, groove 119 is concavely curved. In some embodiments, all or only a portion of groove 119 may be variously configured and dimensioned, such as, for example, planar, concave, polygonal, irregular, uniform, non-uniform, staggered, tapered, consistent or variable.

In some embodiments, instrument 100 includes second body member 130, similar to member 30, including a cup-shaped retaining member 161 having an arcuate inner surface 134 defining a cavity 138 configured to mate with a head 146 on second fastener 148. Cavity 138 is further defined by opposing end surfaces 139, 141 of member 161. Surfaces 139, 141 define an opening 143 therebetween. Opening 143 has a maximum width or diameter that is greater than that of cavity 38 and head 46 has a maximum diameter that is greater than that of cavity 38, as discussed above. This provides a snap-fit configuration in which head 146 can be inserted into cavity 138 in a direction that is perpendicular to axis B1. In particular, member 161 comprises a deformable material that allows a width or diameter of opening 143 to increase as head 146 is positioned through opening 143. Once head 146 passes opening 143, shaft 147 extends through cavity 138 in a direction that is perpendicular or substantially perpendicular to axis B1 and an outer surface of head 146 engages surface 134, thus allowing shaft 147 to rotate relative to member 161 in a plurality of planes.

In some embodiments, cavity 136 and 138 are lowered down vertically onto head 146, snapped together and slid underneath head 126 and 146 respectively, so as to prevent retaining members 160 and 161 from disengaging from heads 126 and 146. Rotating retaining members 160 and 161 to their original vertical position allows heads 126 and 146 to disengage. In some embodiments, member 122 includes a pin 117 extending perpendicular to axis B1 into passageway 140 configured for slidable disposal in groove 119. Groove 119 thus acts as a guide as shaft 116 moves axially through passageway 140. In some embodiments, pin 117 is fixed relative to passageway 140. In some embodiments, pin 117 is removable.

In some embodiments, second body member 130 also includes an inner surface 144 defining a second passageway 142 configured to receive shaft 116 of rack 114. In some embodiments, second passageway 142 is configured to completely or incompletely surround shaft 116 such that shaft 116 is axially translatable along longitudinal axis B1 of shaft 116 from a first end 125 to a second end 128 of shaft 116. In this embodiment, passageway 140 and passageway 142 translate axially along shaft 116 in a movement similar to a caulking gun. In this embodiment, a biasing plate, such as, for example, a spring loaded bar 190, locks shaft 116 in place whenever it stops advancing. Unlike the ratchet mechanism shown in FIGS. 1-4 that moves in predetermined increments, the embodiment shown in FIGS. 5 and 6 moves smoothly along shaft 116 and locks in place whenever spring loaded pressure bar 190 is activated when advancement is stopped. In some embodiments, shaft 116 is configured with a stop 180 that prevents the first or second body 130, 140 from dislodging from shaft 116 as it moves axially along shaft 116.

In operation, to treat a condition of the spine, such as, for example, trauma of the spine, fasteners 124, 148 are inserted into vertebrae such that fasteners 124, 148 are spaced apart from one another. Fasteners 124, 148 are each movable from a first configuration, which may include fasteners 124, 148 being disposed in an initial axial position and in an initial angle relative to one another and a second configuration, which may include fasteners 124, 148 being disposed at a corrected axial position and angle relative to one another.

Member 122 is positioned relative to fastener 124 such that cavity 136 extends perpendicular or substantially perpendicular to an axis defined by shaft 127, similar to cavity 36 and shaft 27 in FIG. 1. Member 122 is rotated about head 126 such that cavity 136 extends perpendicular or substantially perpendicular to the axis defined by shaft 127, as shown in FIGS. 5 and 6, and head 126 engages surface 132 to prevent head 126 from falling through cavity 126 in a manner in which shaft 127 is rotatable relative to member 122 in a plurality of planes.

Spring loaded pressure bar 190 is deactivated such that member 130 is movable along shaft 116 by translating shaft 116 within passageway 142 along axis B1. Member 130 is moved relative to member 122 along shaft 116 a selected distance from member 122 such that cavity 138 is positioned adjacent head 146. In some embodiments, the selected distance correlates to a desired amount of distraction between vertebrae.

Member 130 is positioned relative to fastener 148 such that cavity 138 extends perpendicular or substantially perpendicular to an axis defined by shaft 147, similar to cavity 36 and shaft 27 in FIG. 1. Member 130 is rotated about head 146 such that cavity 138 is coaxial or substantially or substantially coaxial with the axis defined by shaft 147, as shown in FIGS. 5 and 6. Head 146 engages surface 137 to prevent head 146 from falling through cavity 138. Spring loaded pressure bar 190 is reactivated to fix member 130 relative to shaft 116 to maintain the selected distance. This configuration allows a medical practitioner to maintain distraction of vertebrae with instrument 100 while he or she performs an interbody procedure. In some embodiments, instrument 100 self-adjusts to maintain the selected distance, and hence distraction of vertebrae.

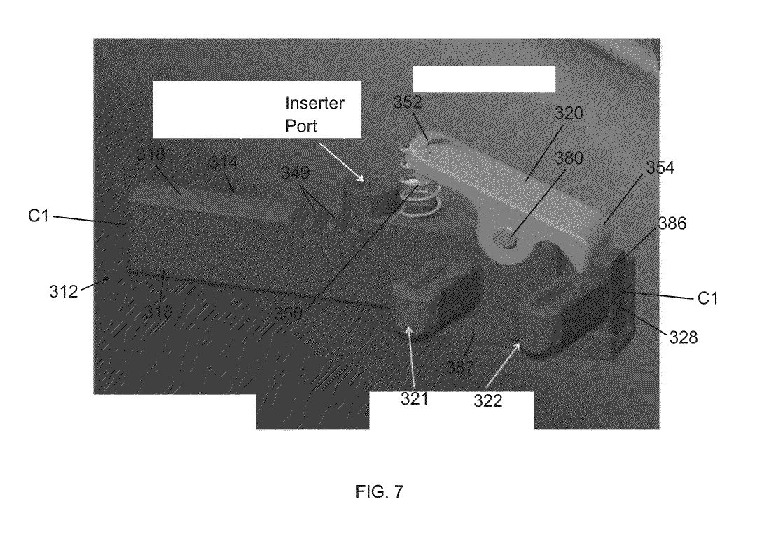

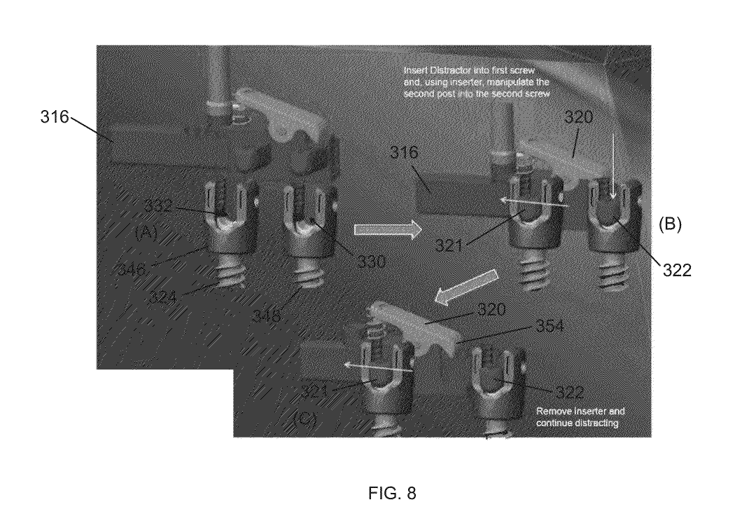

Turning to FIGS. 7 and 8, in some embodiments, system 10 includes an instrument 312 comprising a member, such as, for example, a distraction rack 314. Rack 314 includes a linear shaft 316 defining a longitudinal axis C1. Shaft 316 extends from a first end 326 to a second end 328. Second end 328 comprises a first member 386 including a first body, such as, for example a post 322. Post 322 extends perpendicular to axis C1 and is fixed relative to shaft 316. In some embodiments, post 322 is integrally and/or monolithically formed with shaft 316. Instrument 312 further comprises an outer sleeve 387 that encloses and/or overlaps at least a portion of end 328, as shown in FIGS. 7 and 8, such that shaft 316 is slidably disposed within sleeve 387.

Sleeve 387 includes a second body, such as, for example a post 321 that is fixed relative to sleeve 387 and extends perpendicular to axis C1 such that post 321 is parallel to post 322. Outer sleeve 387 is axially translatable along longitudinal axis C1 of shaft 316 from end 326 to end 328 such that the linear distance between first body 322 and second body 321 increases as outer sleeve 387 moves axially along shaft 316.

In some embodiments, the first and second bodies 321 and 322 have the same or different configurations and have a variously configured cross section configuration, such as, for example, cylindrical, oval, oblong, polygonal, irregular, uniform, non-uniform, variable and/or tapered that fit within the heads of corresponding fasteners. In one embodiment, first and second bodies 321 and 322 have a polygonal configuration and fit within mating surfaces of heads 346 and 326 of fasteners 324 and 348. In some embodiments, first and second bodies 321 and 322 are semi-rigid so as to have a snap-fit configuration with the heads 346 and 326 of corresponding fasteners 324 and 348.

In some embodiments, shaft 316 has a top outer surface 318 that includes a plurality of teeth 349 configured to engage a finger 320, such as, for example a pawl, of a ratchet mechanism of sleeve 387 similar to that described with regard to FIGS. 1-4. The plurality of teeth 349 span completely or partially around the top outer surface 318 of shaft 316. In some embodiments, teeth 349 are uniform but asymmetrical, with each tooth having a moderate slope on one end and a much steeper slope on the other edges of the tooth. In some embodiments, shaft 316 has a variously configured cross section configuration, such as, for example, cylindrical, oval, oblong, triangular, rectangular, square, polygonal, irregular, uniform, non-uniform, variable and/or tapered.

Finger 320 includes a first end 352 and a second end 354. In some embodiments, finger 320 is pivotally connected to sleeve 387 about a pin 380 that extends through opposite side surfaces of sleeve 387 such that end 352 can pivot relative to end 354. A biasing member, such as, for example, a spring 350 is positioned between end 352 and an outer surface of sleeve 387 such that end 354 is biased into engagement with teeth 349. End 354 is configured to engage teeth 349 so that when teeth 349 move axially in an opposite direction of finger 320, end 354 of finger 320 will catch against the steeply slope edge of the first tooth 349 that it encounters, thereby locking finger 320 against the tooth and preventing any further motion. In some embodiments, instrument 312 is a distractor and locking tooth 349 against finger 320 maintains distraction while a surgeon is performing an interbody implant procedure. In some embodiments, the system 10 includes an adjustable distractor configured to maintain a required distance as the surgeon is distracting a vertebral space with different implant trials.

In operation, to treat a condition of the spine, such as, for example, trauma of the spine, fasteners 324, 348 are inserted into vertebrae such that fasteners 324, 348 are spaced apart from one another. Fasteners 324, 348 are each movable from a first configuration, which may include fasteners 324, 348 being disposed in an initial axial position and in an initial angle relative to one another and a second configuration, which may include fasteners 324, 348 being disposed at a corrected axial position and angle relative to one another.

End 328 is positioned relative to fastener 348 such that post 322 is aligned with an implant cavity 330 of fastener 348. A downward force is exerted on end 352 such that end 354 disengages at least one of teeth 349 to allow shaft 316 to slide within sleeve 387 along axis C1. Sleeve 387 is moved relative to end 328 along shaft 316 a selected distance from member end 328 such that post 321 is positioned adjacent head 346 in a manner that aligns post 321 with an implant cavity 332 of fastener 324, as shown in FIG. 8. In some embodiments, the selected distance correlates to a desired amount of distraction between vertebrae. The downward force is removed such that spring 350 pushes end 352 up, causing 354 to engage at least one of teeth 349 to maintain the selected distance, as shown in portion (A) of FIG. 8.

Posts 321, 322 are top-loaded into cavities 330, 332 as shown in portion (B) of FIG. 8. The selected distance may be adjusted by exerting a downward force on end 352 such that end 354 disengages at least one of teeth 349 to allow shaft 316 to slide within sleeve 387 along axis C1. Sleeve 387 is moved relative to end 328 along shaft 316 a second selected distance from end 328 that is different from the first selected distance, as shown in portion (C) of FIG. 8. This configuration allows a medical practitioner to maintain distraction of vertebrae with instrument 312 while he or she performs an interbody procedure. In some embodiments, instrument 312 self-adjusts to maintain the selected distance, and hence distraction of vertebrae.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a member defining a longitudinal axis;
a first body connected to the member and including a surface configured to overlap at least a portion of a first end of a first fastener, the first fastener having a second end configured to penetrate tissue; and
a second body connected to the member and being translatable relative to the first body, the second body including a surface configured to overlap at least a portion of a first end of a second fastener, the second fastener having a second end configured to penetrate tissue.

2. A surgical instrument as recited in claim 1, wherein the first body includes a first cup-shaped retaining member comprising an arcuate inner surface configured to engage a spherical surface of the first end of the first fastener, the inner surface defining a first cavity.

3. A surgical instrument as recited in claim 2, wherein the first cup-shaped retaining member includes opposing end surfaces defining an opening therebetween that is in communication with the first cavity.

4. A surgical instrument as recited in claim 3, wherein a threaded shaft of the first fastener extends through the first cavity when the inner surface engages the spherical surface.

5. A surgical instrument as recited in claim 1, wherein the first body is rotatable about the member and is prevented from translating axially along the member.

6. A surgical instrument as recited in claim 1, wherein the second body includes an inner surface defining a passageway, the second body being movable between a first configuration in which the member is slidably disposed in the passageway and a second configuration in which the second body is fixed relative to the member.

7. A surgical instrument as recited in claim 6, wherein the second body includes a locking mechanism that engages teeth on the member to position the second body in the second configuration.

8. A surgical instrument as recited in claim 7, wherein the locking mechanism is pivotable relative to the member between a first configuration in which a first end of the locking mechanism engages the teeth and a second configuration in which the second end of the locking mechanism is spaced apart from the teeth.

9. A surgical instrument as recited in claim 1, wherein an outer surface of the member is smooth or even.

10. A surgical instrument as recited in claim 9, wherein the member is disposed in a passageway of the second body, the second body being movable between a first configuration in which the member is slidingly disposed in the passageway and a second configuration in which the member is fixed relative to the second body.

11. A surgical instrument as recited in claim 10, wherein a spring loaded pressure bar of the second body is activated to move the member between the first and second positions.

12. A surgical instrument as recited in claim 10, wherein the member includes a stop end configured to prevent the member from being removed from the passageway.

13. A surgical instrument as recited in claim 10, wherein a pin extends through the first body, the pin being disposed in an axial groove of the member such that the groove acts as a guide as the second body moves between the first and second configurations.

14. A surgical instrument as recited in claim 1, wherein the first body is fixed relative to the member and comprises a first post, the second body defining a sleeve having the member slidably disposed therein, the second body comprising a second post.

15. A surgical instrument as recited in claim 14, wherein the first post is parallel to the second post.

16. A surgical instrument as recited in claim 14, wherein the sleeve is movable between a first configuration in which the sleeve is fixed relative to the member and a second configuration in which the member is slidably disposed within the sleeve.

17. A surgical instrument as recited in claim 16, wherein the second body includes a locking mechanism that engages teeth on the member to maintain the sleeve in the first configuration.

18. A surgical instrument as recited in claim 17, wherein the locking mechanism is pivotable relative to the sleeve between a first configuration in which a first end of the locking mechanism engages the teeth and a second configuration in which the first end of the locking mechanism is spaced apart from the teeth.

19. A surgical instrument as recited in claim 18, wherein the locking mechanism is biased to the first configuration of the locking mechanism.

20. A surgical instrument as recited in claim 19, wherein the locking mechanism is biased to the first configuration of the locking mechanism by a spring.
